(19) United States
(12) Patent Application Publication
(10) Pub. No.: US 2005/0100518 A1
(43) Pub. Date: May 12, 2005

(54) Use of modified cellulose derivatives as thickening agent for the organic phase of a nail varnish composition

(76) Inventors: Philippe Ilekti, Maison-Alfort (FR); Jean Mondet, Aulnay -S-Bois (FR); Florence Tournilhac, Paris (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 10/883,819

(22) Filed: Jul. 6, 2004

Related U.S. Application Data
(60) Provisional application No. 60/495,726, filed on Aug. 18, 2003.

(30) Foreign Application Priority Data
Jul. 7, 2003 (FR) 03 50299

(51) Int. Cl. A61K 7/04

(52) U.S. Cl. 424/61

(57) ABSTRACT

The invention relates to the use of certain specific cellulose derivatives chosen from cellulose esters and nitrocelluloses as thickening agents for the organic phase of a nail varnish composition, in particular when the said organic phase comprises at least one ester solvent.

### TECHNICAL FIELD

The present invention relates to the use of novel cellulose derivatives as thickening agents for the organic phase of a nail varnish composition, in particular when the said organic phase comprises ester solvents.

The present invention also relates to nail varnish compositions comprising some of these cellulose derivatives.

### STATE OF THE PRIOR ART

Thickening agents are commonly used in nail varnish compositions for the purpose of thickening the organic phase of these compositions. The thickening of the organic phase makes it possible in particular to improve the suspension of the pigments present in the phase, such as titanium dioxide pigments, and to prevent sedimentation of the latter during storage. The thickening of the organic phase also makes it easier to take the composition out of its packaging and to distribute the said composition over the area to be treated.

Clays, such as bentones, are commonly used to thicken nail varnish compositions. Bentones are clay particles, more particularly hectorite particles, modified at the surface by quaternary alkylammonium chlorides. However, although they contribute a satisfactory shear-thinning nature and a satisfactory thixotropic behaviour to the compositions in which they are incorporated, bentones are however difficult to employ and require in particular a dispersing step. This dispersing step requires a great deal of mechanical energy to wet and to deagglomerate the particles of the bentone, in order to obtain a homogeneous gel comprising a minimum of defects. In addition, the presence of betones in film-forming compositions, such as nail varnishes, modifies the properties of the film obtained after the application of the composition and contributes in particular to reducing the impact strength of the film and to increasing the flaking of the said film. Finally, the fact that the bentones are provided in the form of particles with sizes of the order of a micron produces a matt effect in the film formed and thus results in significant loss in glossiness of this film.

It would therefore be desirable to have available novel thickening agents which do not exhibit the abovementioned disadvantages and which in particular do not contribute to diminishing the glossiness and the strength of the film deposited.

Thus, the authors have discovered, surprisingly, that, by incorporating certain specific cellulose derivatives in a nail varnish composition, it is possible to obtain a composition which exhibits excellent shear-thinning and thixotropic properties, which exhibits a viscosity, in the absence of shearing, which is sufficiently high to prevent sedimentation of the sediments present in the composition, and which, after application, gives films no longer exhibiting the mattness intrinsic to the films obtained from compositions comprising clays as thickening agents.

### DISCLOSURE OF THE INVENTION

Thus, a subject-matter of the invention is the use, as thickening agent for the organic phase of a nail varnish composition, of cellulose derivatives chosen from nitrocelluloses, cellulose esters, the said derivatives comprising free hydroxyl functional groups replaced, in all or part, by radicals of formula —OYR, in which:
R represents a group chosen from:
a) saturated or unsaturated hydrocarbonaceous groups comprising linear or branched chains or saturated or unsaturated cyclic hydrocarbonaceous groups, it being possible for the said groups to comprise, in their chains, one or more aromatic groups and/or one or more heteroatoms chosen from O, N, P, Si or S;
b) fluoro- or perfluoroalkyl groups;
c) groups of polymeric nature chosen from polyolefins, polydienes or polycondensates;
d) organosiloxane or polyorganosiloxane groups;
e) mesogenic groups;
it being possible for the said groups corresponding to the definition a), b), c), d) or e) to comprise at least one group capable of establishing a hydrogen bond,
Y represents a single bond or a divalent bonding group.

Another subject-matter of the present invention is a nail varnish composition comprising an organic phase based on at least one organic solvent and comprising at least one cellulose derivative comprising hydroxyl functional groups replaced, in all or part, by radicals of formula —OYR, in which:
R represents a group chosen from:
a) saturated or unsaturated hydrocarbonaceous groups comprising linear or branched chains or saturated or unsaturated cyclic hydrocarbonaceous groups,
it being possible for the said groups to comprise, in their chains, one or more aromatic groups and/or one or more heteroatoms chosen from O, N, P, Si or S;
b) fluoro- or perfluoroalkyl groups;
c) groups of polymeric nature chosen from polyolefins, polydienes or polycondensates;
d) organosiloxane or polyorganosiloxane groups;
e) mesogenic groups;
it being possible for the said groups corresponding to the definition a), b), c), d) or e) to comprise at least one group capable of establishing a hydrogen bond,
Y represents a single bond or a divalent bonding group,
the said cellulose derivative being a cellulose ester when R corresponds to the definition a), c) and d), a cellulose ester or a nitrocellulose when R corresponds to the definition b), e).

Preferably, for the compositions of the invention, when the cellulose derivative is a cellulose ester, R is chosen from the groups corresponding to the definitions a), b), c) and e) given above.

It is specified that the term “single bond” is understood to mean, previously and subsequently, a single covalent bond forming a bridge between the oxygen and the R group. In this case, the —OYR group corresponds to an —OR group.

It is specified that the term “divalent bonding group” is understood to mean, previously and subsequently, an organic spacer group forming a bridge between the oxygen atom and the R group, it being possible for the said bonding groups to be chosen from the —(C═O)—, —(C═O)O—, —SO₂—, —CO—NH—, —CO—NR′— or —Si(R₃)₂— groups, the R₃ groups, which are identical or different, being a linear or branched hydrocarbonaceous group comprising from 1 to 500 carbon atoms or a cyclic hydrocarbonaceous group comprising from 3 to 500 carbon atoms, the said group being saturated or unsaturated and being able to comprise one or more heteroatoms chosen from O, N, S, Si and/or P, and R′ denoting a C₁ to C₄ alkyl radical. Preferably, R₃ represents an alkyl group comprising from 1 to 10 carbon atoms.

It is specified that the term “nitrocellulose” is understood to mean, previously and subsequently, a polymer composed of an α-(1→4) sequence of partially nitrated anhydroglucose rings which is obtained by esterification of a portion of the free hydroxyl functional groups of a cellulose, for example by the action of nitric acid in the presence of sulphuric acid.

It is specified that the term “cellulose ester” is understood to mean, previously and subsequently, a polymer composed of an α-(1→4) sequence of partially esterified anhydroglucose rings, esterification being obtained by reaction of a portion of the free hydroxyl functional groups of the said rings with a carboxylic acid or a carboxylic acid derivative (acid chloride, acid anhydride). Advantageously, the cellulose esters can be cellulose acetates, propionates, butyrates, isobutyrates, phthalates, acetatobutyrates or acetatopropionates.

Preferably, the organic phase of the varnish composition according to the invention comprises at least one ester solvent, preferably an ester solvent comprising from 3 to 8 carbon atoms, in particular short-chain esters (having from 3 to 8 carbon atoms in total) such as ethyl acetate, methyl acetate, propyl acetate, n-butyl acetate, isobutyl acetate, isopentyl acetate and their mixtures.

The advantage of the cellulose derivatives used in the context of the invention lies in the fact that they exhibit an excellent solubility in the organic phase as defined above of the nail varnish compositions (because of the presence of nitro or ester groups) and that they produce, by virtue of the abovementioned R groups, a thickening of the said organic phase of the said nail varnish compositions and thus prevent sedimentation of the pigments present in the nail varnish compositions of the invention. Furthermore, such derivatives produce, after application to a keratinous substrate of the composition comprising them, glossy and resistant films, in particular resistant to flaking.

According to the invention, the R groups can be groups corresponding to the definition a) given above, namely they can be a saturated or unsaturated hydrocarbonaceous group comprising a linear or branched chain which can comprise from 1 to 50 carbon atoms, preferably from 8 to 50 carbon atoms and which can comprise, in its chain, one or more aromatic groups and/or one or more heteroatoms chosen from O, N, P, Si or S, or can be saturated or unsaturated cyclic hydrocarbonaceous groups, which can comprise from 3 to 50 carbon atoms. It is specified that these groups are non-polymeric in nature, that is to say that they do not result from the polymerization or polycondensation of one or more monomers.

Appropriate groups which are particularly advantageous corresponding to this definition can be linear or branched alkyl groups comprising from 1 to 50 carbon atoms, preferably from 8 to 50 carbon atoms, and optionally comprising one or more heteroatoms chosen from O, N, P, Si or S.

Appropriate groups can also be cycloalkyl groups (for example, mono- or polycyclic) comprising from 3 to 50 carbon atoms.

According to the invention, the R groups can be fluoro- or perfluoroalkyl groups (definition b)), that is to say alkyl groups in which all or part (all when the group is perfluoro) of the hydrogen atoms are substituted by fluorine atoms. Preferably, these fluoro- or perfluoroalkyl groups comprise from 6 to 50 carbon atoms.

According to the invention, the R groups can also be groups of polymeric nature chosen from polyolefins, polydienes or polycondensates (definition c)).

Appropriate polyolefins can be polyethylene, advantageously comprising from 10 to 500 —CH₂— units, or copolymers chosen from the following copolymers:
(ethylene/propylene) copolymers;
(ethylene/butene) copolymers;
(ethylene/hexene) copolymers;
(ethylene/octene) copolymers.

Examples of polydienes are polybutadienes or polyisoprene, which are preferably hydrogenated.

According to the invention, the polycondensates can be polyesters, polyamides, polyesteramides, polyurethanes, polycarbonates, polyureas, (urea/urethane) copolymers or polyethers.

Mention may be made, as examples of polyesters, of those resulting from the polyesterification between diols, such as ethylene glycol, propylene glycol, diethylene glycol, neopentyl glycol, 1,4-butanediol, furandimethanol, cyclohexanedimethanol, glycerol, trimethylolpropane, pentaertythritol and their mixtures, and polycarboxylic acids, in particular dicarboxylic acids, and their C₁-C₄ ester derivatives, for example succinic acid, glutaric acid and adipic acid or their dimethyl esters, phthalic anhydride or dimethyl terephthalate, or lactones, for example caprolactone.

Mention may be made, as examples of polyesteramides, of those obtained by addition of amino alcohols, such as ethanolamine, to polyesterification mixtures, such as those mentioned above.

Mention may be made, as examples of polycarbonates, of those obtained by reaction between diols, such as 1,3-propanediol, 1,4-butanediol, 1,8-hexanediol, diethylene glycol or tetraethylene glycol, with diaryl, diacyl or aliphatic carbonates, for example diphenyl carbonate, or with phosgene.

Mention may be made, as examples of polyamides, of those obtained by condensation between an aliphatic, cycloaliphatic or aromatic C₃-C₅₀ dicarboxylic acid (or C₁-C₄ ester derivative) and a linear or branched aliphatic, cycloaliphatic or aromatic C₂-C₅₀ diamine, it being possible for the diacids to be chosen from the diacids mentioned above with, in addition, dimeric fatty acids (originating from the condensation between two molecules of unsaturated fatty monoacids), it being possible for the diamines to be chosen from ethylenediamine, 1,2-diaminopropane, 1,3-diaminopropane, 1,4-diaminobutane, 1,2-diamino-2-methylpropane, 1,6-diaminohexane, 1,10-diaminodecane, isophoronediamine, adamantanediamine or 2,6-diaminopyridine.

Mention may be made, as examples of polyurethanes, polyureas and polyureas-urethanes, of those obtained by polyaddition between aliphatic, cycloaliphatic and/or aromatic C₄-C₁₀₀ , preferably C₄-C₃₀, diisocyanates, such as hexamethylene diisocyanate, isophorone diisocyanate, toluene diisocyanate or diphenylmethane diisocyanate, and diols, such as defined above, or diamines, such as defined above, or diols/diamines mixtures.

Mention may be made, as examples of polyethers, of copolymers between oxyethylene and oxypropylene or of polytetramethylene oxide.

For the compositions of the invention, when the cellulose derivative is a cellulose ester and when Y represents a group of formula —CO—, then the groups R corresponding to the definition a) or c) are prerably different from those preexisting on the cellulose ester before replacement of hydroxyl functional groups, in all or part, by radicals of formula —OYR (these preexisting groups being hydrocarbonaceous groups, for example, such as the cellulose ester, before the grafting of groups R as defined above, is cellulose acetate, propionate, butyrate, isobutyrate, phthalate, acetatobutyrate or acetatopropionate).

The R groups can also be organosiloxane or polyorganosiloxane groups (definition d)). Such groups can correspond to the following formula:
—[Si(Ra,Rb)—O—]ₚ—Si(Ra,Rb,Rc),
in which:
the Ra, Rb and Rc groups, which are identical or different, represent a linear or branched alkyl chain comprising from 1 to 20 carbon atoms, a phenyl or a perfluoroalkyl chain comprising from 3 to 20 carbon atoms, and p is an integer ranging from 0 to 500.

Finally, the R groups can represent mesogenic groups, that is to say groups capable of giving liquid crystal phases.

Mention may be made, among the mesogenic groups corresponding to the definition given above capable of giving liquid crystal phases resulting in gelling, of alkylaryl, alkylcycloalkyl, alkoxyaryl or alkoxycycloalkyl groups.

Advantageously, such groups corresponding to the definition given above can correspond to the following formulae: 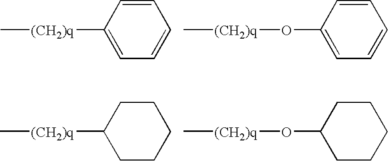
with q being an integer ranging from 1 to 10.

Mention may advantageously be made, among the mesogenic groups corresponding to the definition given above capable of being suitable, of the groups represented by the following general formula:
-E-F-E-G,
in which:
the E groups, which are identical or different, are divalent aromatic groups, such as: 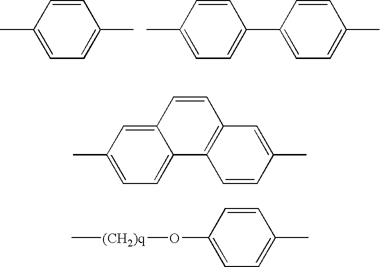
with q representing an integer ranging from 1 to 10;
F is a divalent unsaturated and non-cyclic group such as:
   —C═C—, —N═N—, —CH═N—, —CO—O—
   or a single bond;
G represents an end group of following formulae:
   —O—Ru, —CO—Ru, —CN, —COOH, —(CH₂)_{q}—Ru
   with Ru representing a linear or branched alkyl group comprising from 1 to 10 carbon atoms and q representing an integer ranging from 1 to 10.

Specific groups corresponding to this definition are the groups of following formulae:

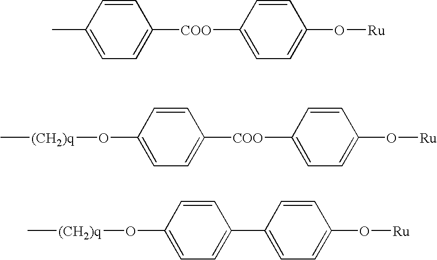

Mention may also be made, among the mesogenic groups capable of being suitable, of the para-hydroxybenzoic acid derivatives corresponding to one of the following formulae: 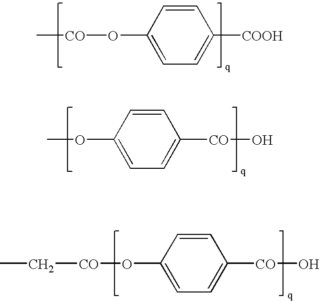
with q having the same definition as that given above.

Finally, mention may be made, as advantageous mesogenic group, of groups derived from cholesterol, such as that represented by the following formula: 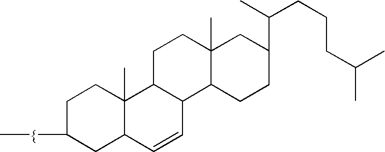
the bonding with Y or O (when Y represents a single bond) taking place at the bond interrupted by the bracket.

Advantageously, the groups corresponding to the definitions a), b), c), d) and e) can carry one or more groups capable of establishing a hydrogen bond.

It is specified that the term “group capable of establishing a hydrogen bond” is understood to mean a group comprising either a hydrogen atom bonded to an electronegative atom or an electronegative atom. When the group comprises a hydrogen atom bonded to an electronegative atom, the hydrogen atom can interact with another electronegative atom carried, for example, by another molecule, such as the keratin of the nail, to form a hydrogen bond. When the group comprises an electronegative atom, the electronegative atom can interact with the hydrogen atom bonded to an electronegative atom carried, for example, by another mole, such as the keratin of the nail, to form a hydrogen bond.

Advantageously, these groups capable of establishing a hydrogen bond can be groups chosen from the following groups:
hydroxyl —OH;
carboxylic acid —COOH;
amino —NR₁R₂ with R₁ and R₂ identical or different;
pyridino of formula: 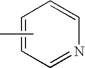
pyrimidino of formula: 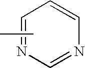
oxazolino corresponding to one of the following formulae: 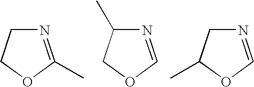
amido of formulae —NH—CO—R′ or —CO—NH—R₁;
pyrrolidono corresponding to one of the following formulae: 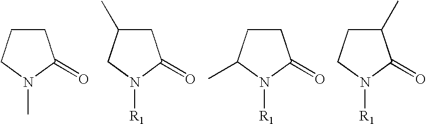
carbamoyl of formulae —O—CO—NH—R′ or —NH—CO—O—R′;
thiocarbamoyl of formulae —O—CS—NHR₁ or —NH—CS—O—R′;
carbonate —O—CO—O—R′;
ureyl —NR₁—CO—N(R₁)₂, the R₁ groups being identical or different;
thioureyl —NR₁—CS—N(R₁)₂, the R₁ groups being identical or different;
oxamido —NRP—CO—CO—N(R₁)₂ with the R₁ groups identical or different;
guanidino —NH—C(═NH)—N(R₁)₂ with the R₁ groups identical or different;
biguanidino —NH—C(═NH)—NH—C(═NH)—N(R₁)₂ with the R₁ groups identical or different;
sulphonamido —NR₁—S(═O)₂—R′, with R₁ and R₂ representing H or an alkyl group comprising from 1 to 4 carbon atoms, R′ representing an alkyl radical comprising from 1 to 4 carbon atoms.

It is understood that such groups are carried by the R chain either at the chain end or in the side position with respect to the said chain.

Derivatives carrying at least one group capable of establishing a hydrogen bond are particularly advantageous as they contribute, to the nail varnish compositions comprising them, very good adhesive properties by virtue of the ability of these groups to establish a hydrogen bond, for example with the keratin of the nail.

Preferably, the R groups corresponding to the definitions a), b), c), d) or e) are not charged, i.e are not anionic, cationic, amphoteric (i.e carrying both one negative charge and one cationic charge) or betaine.

As was mentioned above, the R groups grafted to the hydroxyl functional groups of the cellulose derivatives confer thickening properties on these derivatives, in particular when these derivatives are incorporated in nail varnish compositions comprising an organic phase, in particular an organic phase based on ester solvent(s).

The mechanism responsible for the thickening effect varies according to the nature of the R groups grafted to the cellulose derivatives.

Thus, a first mechanism can consist of a phase separation between the R groups and the backbone of the cellulose derivatives. The phase separation is due to an incompatibility (insolubility) between the backbone of the cellulose derivatives carrying ester or nitrate groups (rather polar) and the side R groups (very non polar in the case of organosiloxanes) (the grafted side groups are incompatible both with the backbone of the cellulose derivatives and with the solvent). This mechanism is encountered in the case where R represents an organosiloxane or polyorganosiloxane group, such as those of following formulae:
—[Si(Ra,Rb)—O—]ₚ—Si(Ra,Rb,Rc),
in which:
the Ra, Rb and Rc groups, which are identical or different, represent a linear or branched alkyl chain comprising from 1 to 20 carbon atoms, a phenyl or a perfluoroalkyl chain comprising from 3 to 20 carbon atoms, and p is an integer ranging from 0 to 500.

A second mechanism responsible for the thickening effect of the cellulose derivatives can consist of a crystallization of the groups carried by R in the presence, for example, of the ester solvents of the organic phase of the nail varnish compositions.

Suitable mechanisms can be the case:
of polyolefin grafts, such as polyethylene or (ethylene/propylene), (ethylene/butene), (ethylene/hexene) or (ethylene/octene) copolymers;
of linear perfluoroalkyl groups comprising from 6 to 30 carbon atoms;
of polyester grafts, such as aliphatic polyesters, for example polycaprolactone of formula —[(CH₂)₅—C(═O)ᵣ—]ᵣ—R_{d} with r being an integer ranging from 10 to 500 and Rd representing a linear or branched alkyl group comprising from 1 to 20 carbon atoms or a cyclic group comprising from 3 to 20 carbon atoms; or of aromatic polyesters, such as poly(ethylene terephthalate) and poly(butylene terephthalate) of formulae: 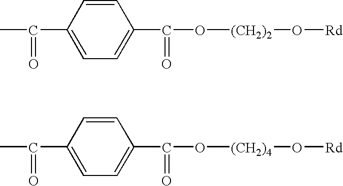
with Rd corresponding to the same definition as that given above.

Another mechanism responsible for the thickening effect of the cellulose derivatives lies in the creation of hydrogen interactions between the groups carried by R, it being possible for the said interactions to take place within the same molecule (“intramolecular interactions”) or between different molecules (“intermolecular interactions”), or similarly in the creation of acid-based interactions within the Lewis meaning. Such a mechanism is the case of the R groups carrying groups capable of establishing hydrogen bonds and of the polymeric R groups, such as polyamides, polyurethanes and polyureas.

Finally, a mechanism responsible for the thickening effect of the cellulose derivatives can lie in an organization of “liquid crystal” type of the appropriate grafted R groups. This is the case when the R groups represent mesogenic groups, more particularly thermotropic mesogenic groups of smectic or nematic type, that is to say that they will arrange themselves in the form of layers (“smectic”) or of strings (“nematic”), which will bridge the cellulose molecules and will trap the solvents, in particular esters, of the organic phase of the nail varnish compositions in a three-dimensional network.

Appropriate R groups can be groups of general formula:
-E-F-E-G,
in which:
the E groups, which are identical or different, are divalent aromatic groups, such as: 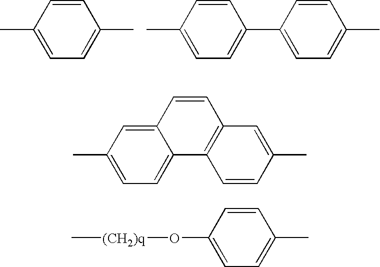
with q representing an integer ranging from 1 to 10;
F is a divalent unsaturated and non-cyclic group such as:
   —C═C—, —N═N—, —CH═N—, —CO—O—
   or a single bond;
G represents an end group of following formulae:
   —O—Ru, —CO—Ru, —CN, —COOH, —(CH₂)_{q}—Ru
   with Ru representing a linear or branched alkyl group comprising from 1 to 10 carbon atoms and q representing an integer ranging from 1 to 10.

Specific groups corresponding to this definition are the groups of following formulae: 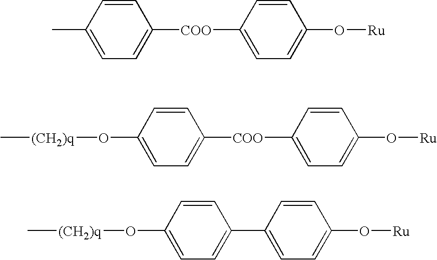
R groups to which this type of mechanism relates can also be groups derived from para-hydroxybenzoic acid corresponding to one of the following formulae: 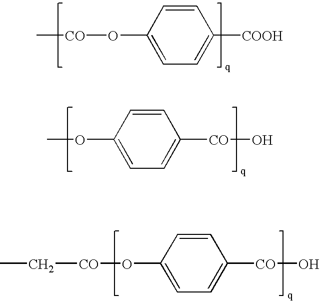
R groups to which this type of mechanism relates can also be cholesterol derivatives, such as the group of following formula:

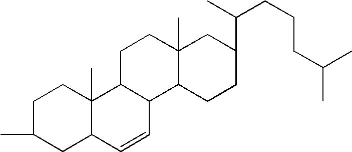

Finally, R groups to which this type of mechanism relates can be groups corresponding to the following formulae: 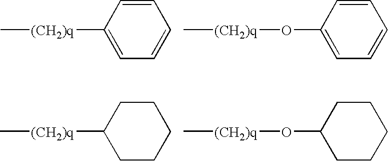
with q being an integer ranging from 1 to 10.

As was indicated above, the modified cellulose derivatives described above are thickening agents for the organic phase of nail varnish compositions, more particularly when the organic phase comprises ester solvents. These derivatives can also, in addition to their thickening role, provide the nail varnish compositions in which they are incorporated with a film-forming role, it being understood that the abovementioned derivatives will be incorporated in a larger amount when they are intended, in addition to the thickening role, to perform a film-forming role.

Preferably, the compositions of the invention are compositions able to form a film without needing thermical, chemical, photochemical crosslinking and are advantageously free of crosslinking agents and crosslinking initiators, such as photoinitiators. In other words, the compositions of the invention are able to form a non-crosslinked film by only the application of the composition on a substrate followed by the evaporation of the solvent(s), the said film being removable by classical removers, for example, used in the field of nail varnish compositions.

The modified cellulose derivative or derivatives in accordance with the invention can represent from 0.1 to 60%, preferably from 0.5 to 40% and better still from 1 to 30% by weight, with respect to the total weight of the nail varnish composition.

Preferably, when the modified cellulose derivatives are used both as thickening agents and as film-forming agents, the content of these derivatives in the nail varnish composition ranges from 10 to 40%, preferably from 20 to 35%, by weight with respect to the total weight of the composition.

Preferably, when the modified cellulose derivatives are used solely as thickening agents, the content of these derivatives in the nail varnish composition ranges from 0.1 to 20%, preferably from 1 to 10%, by weight with respect to the total weight of the composition.

The nail varnish compositions in which the cellulose derivatives described above are incorporated can be employed as varnish base, as product for making up the nails, as finishing composition also known as topcoat, to be applied to the product for making up the nails, or alternatively as product for the cosmetic care of the nails. These compositions can be applied to the nails of human beings or alternatively to false nails.

The nail varnish compositions in which the cellulose derivatives of the invention are incorporated can comprise, in addition to ester solvents, one or more solvents chosen from:
ketones which are liquid at ambient temperature, such as methyl ethyl ketone, methyl isobutyl ketone, diisobutyl ketone, isophorone, cyclohexanone or acetone;
alcohols which are liquid at ambient temperature, such as ethanol, isopropanol, diacetone alcohol, 2-butoxyethanol or cyclohexanol;
glycols which are liquid at ambient temperature, such as ethylene glycol, propylene glycol, pentylene glycol or glycerol;
propylene glycol ethers which are liquid at ambient temperature, such as propylene glycol monomethyl ether, propylene glycol monomethyl ether acetate or dipropylene glycol mono(n-butyl) ether;
short chain esters (having from 3 to 8 carbon atoms in total), such as ethyl acetate, methyl acetate, propyl acetate, n-butyl acetate or isopentyl acetate;
ethers which are liquid at ambient temperature, such as diethyl ether, dimethyl ether or dichlorodiethyl ether;
alkanes which are liquid at ambient temperature, such as decane, heptane, dodecane or cyclohexane;
aromatic cyclic compounds which are liquid at ambient temperature, such as toluene and xylene; and their mixtures.

The content of organic solvent(s) (ester solvent and/or additional solvent) in the nail varnish composition can range from 20 to 90%, preferably from 30 to 80% and better still from 40 to 70% by weight, with respect to the total weight of the composition.

The composition comprising an organic phase can additionally comprise water, in particular in a content ranging from 0.1 to 10% by weight with respect to the total weight of the composition, preferably less than 2% of water by weight.

The composition can also comprise one or more additives chosen from film-forming agents, plasticizers, colouring materials, such as pigments, pearlescent agents, glitter, thickening agents other than the cellulose derivatives as described above, spreading agents, wetting agents, dispersing agents, antifoaming agents, preservatives, UV-screening agents, active principles, surfactants, waxes, moisturising agents, fragrances, neutralizing agents, stabilizing agents or antioxidants.

Thus, the composition can comprise an additional film-forming polymer which can be chosen from nitrocelluloses, cellulose esters, radical polymers, polycondensates and polymers of natural origin, and their blends.

The additional film-forming polymer can be chosen in particular from the group formed by vinyl polymers, polyurethanes, polyesters, alkyd resins, epoxy ester resins, nitrocelluloses, cellulose esters, such as cellulose acetates, propionates, butyrates, isobutyrates, acetate/propionates or acetate/butyrates, the resins resulting from the condensation of formaldehyde with an arylsulphonamide, and their blends.

The additional film-forming polymer can be present in a content ranging from 0.1% to 40% by weight with respect to the total weight of the composition and preferably ranging from 1% to 35% by weight.

The composition can additionally comprise at least one plasticizer. Mention may in particular be made, alone or as a mixture, of conventional plasticizers, such as:
glycols and their derivatives, such as diethylene glycol ethyl ether, diethylene glycol methyl ether, diethylene glycol butyl ether or diethylene glycol hexyl ether, ethylene glycol ethyl ether, ethylene glycol butyl ether or ethylene glycol hexyl ether;
glycerol esters,
propylene glycol derivatives and in particular propylene glycol phenyl ether, propylene glycol diacetate, dipropylene glycol butyl ether, tripropylene glycol butyl ether, propylene glycol methyl ether, dipropylene glycol ethyl ether, tripropylene glycol methyl ether and diethylene glycol methyl ether, or propylene glycol butyl ether,
esters of acids, in particular carboxylic acids, such as citrates, phthalates, adipates, carbonates, tartrates, phosphates or sebacates,
oxyethylenated derivatives, such as oxyethylenated oils, in particular vegetable oils, such as castor oil;
their mixtures.

The amount of plasticizer can be chosen by a person skilled in the art on the basis of his or her general knowledge such as to obtain a composition having cosmetically acceptable properties. The content of plasticizer can, for example, range from 0.1% to 15% by weight with respect to the total weight of the composition and preferably from 0.5% to 10% by weight.

The composition can comprise a colouring material which can be chosen from pulverulent compounds and/or dyes which are soluble in the medium of the composition. The colouring material can be present in a content ranging from 0.001% to 10% by weight with respect to the total weight of the composition. The pulverulent compounds can be chosen from pigments and/or pearlescent agents and/or glitter commonly used in nail varnishes.

The pigments can be white or coloured and inorganic and/or organic. Mention may be made, among inorganic pigments, of titanium dioxide, optionally surface-treated, zirconium or cerium oxides, iron or chromium oxides, manganese violet, ultramarine blue, chromium hydrate, ferric blue or metallic pigments, such as aluminium or bronze. Mention may be made, among organic pigments, of carbon black, pigments of D & C type, barium, strontium, calcium and aluminium lakes based on cochenille carmine, or guanine.

The pearlescent pigments can be chosen from white pearlescent pigments, such as mica covered with titanium oxide or with bismuth oxychloride, coloured pearlescent pigments, such as titanium oxide-coated mica, with iron oxides, titanium oxide-coated mica with in particular ferric blue or chromium oxide, or titanium oxide-coated mica with an organic pigment of the abovementioned type, and pearlescent pigments based on bismuth oxychloride.

The glitter can be chosen from that made of poly(ethylene terephthalate), polyester or acrylic resin or made of aluminium.

The dyes are for example, Sudan red, DC Red 17, DC Green 6, β-carotene, soybean oil, Sudan brown, DC Yellow 11, DC Violet 2, DC Orange 5 or quinoline yellow.

The composition according to the invention can additionally comprise any additive known to a person skilled in the art as being capable of being incorporated in such a composition, such as thickening agents, spreading agents, wetting agents, dispersing agents, antifoaming agents, preservatives, UV-screening agents, active principles, surfactants, waxes, moisturising agents, fragrances, neutralizing agents, stabilizing agents or antioxidants. Of course, a person skilled in the art will take care to choose this or these optional additional compounds and/or their amounts so that the advantageous properties of the composition for the use according to the invention are not, or not substantially, detrimentally affected by the envisaged addition.

The cellulose derivatives used as thickening agents according to the invention can be commercially available derivatives but can also be prepared by various processes within the scope of a person skilled in the art and in particular according to the following two main synthetic routes:
either the starting materials are celluloses already modified by nitro groups or ester groups and these celluloses, thus modified, are reacted with reactants appropriate for grafting, to the free hydroxyl functional groups, “thickening” groups of formula —Y—R as defined above (referred to in the continuation of this description as Route A);
or the starting materials are celluloses already modified by —O—Y—R groups as defined above and these celluloses, thus modified, are reacted with reactants appropriate for obtaining the nitration or the esterification of at least a portion of the free hydroxyl functional groups (referred to as Route B).

The preferred route for the synthesis of the cellulose derivatives of the invention is Route A. Only this route will form the subject of a detailed description in the present application.

According to this synthetic route, the starting celluloses can be nitrocelluloses or cellulose esters comprising a certain number of free OH functional groups with which appropriate reactants will react to give —O—Y—R groups.

The starting cellulose derivatives used to introduce the R groups as defined above can be film-forming nitrocelluloses commonly used in inks, paints and nail varnish formulations. These nitrocelluloses are prepared industrially by esterification of cellulose with a mixture of nitric acid and sulphuric acid, the latter acting as dehydrating agent and shifting the esterification equilibrium. They thus constitute cellulose nitrates, commonly denoted under the name of nitrocellulose.

Preferably, the starting nitrocelluloses exhibit a percentage of nitrogen of 10 to 13.5% by weight (a fully nitrated cellulose having a percentage of nitrogen of 14.14%), which corresponds substantially to a level of nitration of 1.7 to 2.5 esterified hydroxyl groups per anhydroglucose ring out of the three free —OH groups of the ring initially available for nitration. In other words, between 1 and 0.75 free —OH group remains for the grafting of R groups per anhydroglucose ring of the nitrocellulose.

As regards the molecular weight of the starting nitrocelluloses, the weight is expressed generally by the measurement of the viscosity (by ball drop) of a solution of the polymer (or collodion) in a mixture of solvents at a given % of polymer, the said percentage of polymer generally being from 12 to 25%. In general, the mixture of solvents used for the characterization is composed of 25% denatured 95% ethanol, 20% ethyl acetate and 55% toluene, the percentages being expressed by weight with respect to the mixture of solvents.

Preferably, the starting nitrocelluloses used to prepare the nitrocelluloses modified according to the invention have ball drop viscosities ranging from 1/16 to 1000 seconds, preferably ¼ to 200 seconds, for a concentration of polymer of 12.2% in the abovementioned mixture of solvents.

From the packaging view point, the starting nitrocelluloses are generally, because of their flammable nature and their explosive power, presented:
either in a form wetted with, for example, 35% of alcohol (ethanol, isopropanol);
or in solution in a solvent devoid of labile hydrogen;
or in the form of chips, that is to say of a mixture comprising, for example, 80% of the polymer and 20% of a plasticizer, such as dibutyl phthalate.

In view of the dangers in handling the starting nitrocellulose, the processes for the preparation of the nitrocelluloses modified according to the invention will be carried out under mild conditions, namely:
by operating at a reaction temperature of 0 to 80° C., preferably of 20 to 60° C.;
by operating, preferably, in the absence of oxygen, to avoid any risk of explosion by contact of the nitrocellulose with an oxidizing agent, for example by operating under an inert gas, such as argon.

The starting cellulose esters used to graft R groups as defined above are cellulose derivatives exhibiting a degree of esterification sufficient to confer, on the resulting derivatives, a solubility in the organic phase of nail varnish compositions, in particular when the organic phase comprises one or more ester solvents.

Whether nitrocelluloses or cellulose esters as defined above are used as base reactant, the reaction for grafting —Y—R groups as defined above differs according to whether R is or is not of polymeric origin.

When R is a group of non-polymeric origin (that is to say when R corresponds to the definitions a), b), d) and e)), the grafting reaction can be carried out in an inert solvent by reaction chosen from the following reactions: etherification, esterification with a carboxylic acid or its derivatives, transesterification with an ester or a carbonate, esterification with a sulphonic acid or its derivatives, reaction with an isocyanate or reaction with an alkoxysilane.

For the parts which follow, in the account of the methods for the preparation of the cellulose derivatives (nitrocellulose of cellulose ester) modified according to the invention, use will be made of the following abbreviations:
Cell-OH for the unmodified starting cellulose derivative (nitrocellulose or cellulose ester), a single OH being taken into account in the interests of clarity of the description;
R for the group to be grafted to the nitrocellulose, corresponding to the definitions given above.

In the part hereinafter, a description will be given of different reactions which can be used for the grafting of R groups of non-polymeric nature (that is to say, corresponding to the definitions a), b), d) and e)).

1) Etherification

The following reactions can be envisaged for the etherification, reactions for which the Y radical forming the junction between the R groups and the nitrocellulose is a single bond:
reaction with an alkyl halide R—X (X representing a halogen) in a basic medium (for example in the presence of an aqueous sodium hydroxide solution);
   Cell-OH+R—X→Cell-OR+HX
   with X being a halogen chosen from chlorine, bromine or iodine;
reaction with an epoxide: 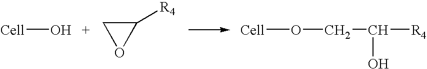
with R₄ representing a group participating in the constitution of the R group defined above, the said R group being represented here by the —CH₂—(CHOH)—R₄ group; appropriate epoxide reactants can be 1,2-epoxyoctane, 1,3-epoxynonane, 1,2-epoxydecane, 1,2-epoxyneodecane, 1,2-epoxycyclodecane, 1,2-epoxy-cyclododecane, 1,2-epoxycyclohexane or 1,2-epoxy-3-phenoxypropane;
reaction between an aldehyde in a reducing medium (such as triethylsilane in the presence of platinum): 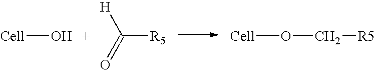
with R₅ representing a group participating in the constitution of the R group, the said R group being represented here by the —CH₂—R₅ group.

According to an alternative form, this reaction can take place in two steps with, for the first step, a preliminary reaction of the aldehyde with a diol, such as glycol, to form a cyclic acetal: 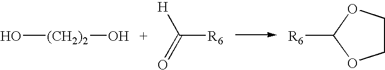
followed by a reaction of the cyclic acetal with the nitrocellulose: 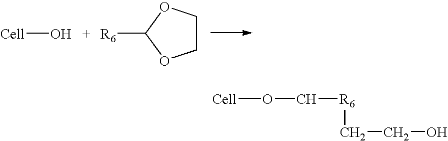
with R₆ representing a group participating in the constitution of the R group, the said R group being represented here by the —CH—(CH₂-CH₂—OH)—R₆ group;
reaction with a mixed ether R—O—R′, with R′ denoting a C₁-C₄ alkyl radical, in an acidic medium:
   Cell-OH+R′—O—R→Cell-O—R+R′—OH
addition reaction of the free —OH groups of the nitrocellulose or cellulose ester to a double bond, for example an end double bond, carried by the radical to be grafted, in the presence of PdCl₂ and HgCl₂:
   Cell-OH+CH₂═CH—R₇→Cell-O—CH₂—CH₂—R₇
   or
   Cell-OH+Rₐ—CH═CH—Rb→Cell-O—CHRₐ—CH₂—R_{b}
R₇ representing a group participating in the constitution of the R group, represented here by the —CH₂—CH₂—R₇ group, and Rₐ and R_{f} representing a group participating in the constitution of the R group, represented here by the —CHRₐ—CH₂—R_{b} group.

Other etherification reactions can be envisaged, in particular those mentioned in the work “Advanced Organic Chemistry”, J. March, John Wiley & Son, 1992 Edition.

2) Esterification

Mention may be made, as examples for the esterification reactions, of the following reactions, reactions for which Y represents a —CO— divalent bonding group:
reaction with a carboxylic acid R—CO₂H:
   Cell-OH+R—CO₂H→Cell-O—CO—R+H₂O
reaction with an acid chloride R—COCl or transesterification by an ester R—COOR′, such as:
   Cell-OH+R—COCl→Cell-O—CO—R+HCl
   Cell-OH+R—COO—R′→Cell-O—CO—R+R′—OH
reaction with an acid anhydride, for example: 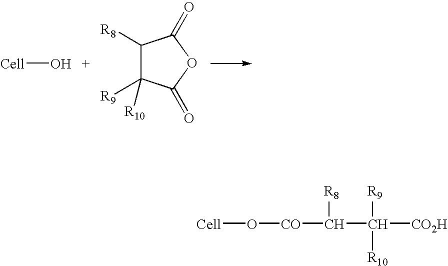
with R₈, R₉ and R₁₀ being such that —(CHR₈)—CR₉R₁₀—CO₂H represents R.

Appropriate esterification reactants can be: octanoic acid, 2-ethylhexanoic acid, nonanoic acid, decanoic acid, neodecanoic acid, undecanoic acid, dodecanoit acid, isononanoic acid, palmitic acid, octadecanoic acid, behenic acid and their acid chloride or acid anhydride derivatives.

3) Transesterification with a Carbonate

The following reaction with a carbonate R′—O—CO—O—R can be envisaged, reaction for which Y represents a —CO—O— connecting group:
Cell-OH+R′—O—CO—O—R→Cell-O—CO—O—R+R′—OH
4) Esterification with a Sulphonyl Chloride

The following reaction can be envisaged as examples for the esterification reactions with a sulphonic acid or a sulphonyl chloride, reactions for which Y represents a —SO₂— divalent bonding group:
Cell-OH+Cl—SO₂—R→Cell-O—SO₂—R+HCl
5) Reaction with an Isocyanate

The following reaction with an isocyanate OCN—R can be envisaged as examples for the reactions for the formation of carbamate bonds, reactions for which Y represents a —CO—NH— divalent bonding group:
Cell-OH+OCN—R→Cell-O—CO—NHR

Appropriate isocyanate reactants can be butyl, isobutyl, pentyl, hexyl, heptyl, octyl, 2-ethylhexyl, nonyl, decyl, undecyl, dodecyl or phenyl isocyanate.

6) Reaction with an Alkoxysilane

The following reaction can be envisaged as examples for the reactions with an alkoxysilane, reactions for which Y represents a —Si(R₁₂)₂— divalent bonding group: 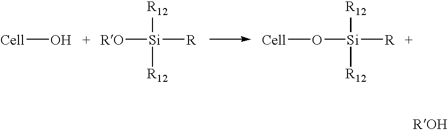
with it being possible for the R₁₂ groups, which are identical or different, to be a linear or branched hydrocarbonaceous group comprising from 1 to 500 carbon atoms or a cyclic hydrocarbonaceous group comprising from 3 to 500 carbon atoms, the said group being saturated or unsaturated and being able to comprise one or more heteroatoms chosen from O, N, S, Si and/or P, the R₁₂ groups thus having the same definition as the R₃ groups defined above.

When R is a group of polymeric origin, the grafting reaction can be carried out according to the following scheme:
Cell-OH+X₁—POL→Cell-O—Y—POL
with POL representing the polymer, the sequence of which corresponds to the definition of the R group given above (definition c), X₁ representing a functional group carried by the polymer, the said functional group being reactive with respect to the hydroxyls of the starting cellulose derivative (nitrocellulose or cellulose ester), and Y corresponding to the same definition as that given above and resulting from the reaction of —OH with X₁.

In the same way as for the grafting of R groups of non-polymeric nature, the functional groups X₁ which are reactive with respect to the free hydroxyl functional groups of the starting cellulose derivative can be chosen from epoxide, aldehyde, acetal, halogen (chlorine, bromine, iodine), ethylene, carboxylic acid or derivative (chloride, anhydride, C₁-C₄ alkyl ester), carbonate, sulphonic acid or sulphonyl chloride, isocyanate or monoalkoxysilane functional groups.

However, in view of the dangers of the handling of nitrocellulose which is mentioned above, the reactive functional groups X₁ of the polymers used in the grafting reaction are preferably chosen from carboxylic acid chloride, carboxylic acid anhydride, carboxylic acid (in the presence of a coupling reagent of the DCCI type), monoisocyanate, monoalkoxysilane, monoepoxide, halogen (Cl, Br, I), C₁-C₄ alkyl monoether or vinyl double bond functional groups.

The starting POL—X₁ polymers have to be synthesized, apart from those for which X₁ is a vinyl-type reactive double bond, many of which are commercially available. The POL—X₁ polymers can be synthesized, for example, from a polymer comprising a reactive functional group different from X₁ which is converted by appropriate conventional reactions to X₁.

Mention may be made, as examples, of the following reaction: 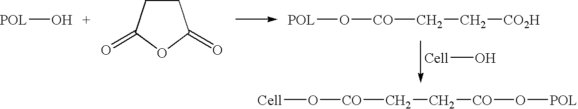
According to a second implementation, the grafting reaction can consist, in a first step, in converting all or part of the hydroxyl functional groups of the starting cellulose derivative (nitrocellulose or cellulose ester) to reactive functional groups and then, in a second step, in reacting the said reactive functional groups with appropriate reactive ends of polymers comprising the said R group.

Mention may be made, as examples, of the following reaction:

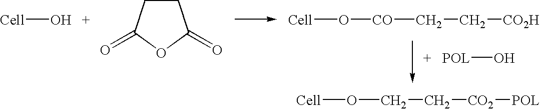

The reaction of Cell-OCOCH₂CH₂—CO₂H with Cell-OH can take place but conditions are chosen so that this reaction is minimized (crosslinking is prevented).

According to the invention, the polymer grafts can be:
polyolefins (homo- or copolymers), preferably semi-crystalline polyolefins;
polydienes, preferably hydrogenated polydienes;
polycondensates, such as:
semi-crystalline aliphatic polyesters, such as polycaprolactone, or semi-crystalline aromatic polyesters, such as poly(ethylene glycol terephthalate) or poly(butylene glycol terephthalate);
polyamides;
polyurethanes, polyureas or (urea/urethane) copolymers;
polyorganosiloxanes.

Mention may be made, as examples of polyolefins, of the polymers obtained by homopolymerization or copolymerization of monomers chosen from:
α-olefins, for example C₂ to C₂₀ α-olefins, in particular α-olefin copolymers, the monomers of which give crystalline homopolymers, and homopolymers or copolymers of branched α-olefins. Mention may in particular be made of isobutylene homopolymer and copolymers of ethylene (or propylene) and of longer α-olefins, such as butene, hexene, octene, decene or dodecene. Mention may also be made, for these α-olefins, of their noncrystalline copolymers with cycloolefins, in particular the copolymers between ethylene (or propylene) and norbornene or norbornene derivatives;
dienes, for example C₄ to C₂₀ dienes, such as butadiene, isoprene, hexadiene, and the like, which give copolymers with other vinyl monomers, such as the a-olefins mentioned above, and, in addition, with styrene or substituted styrenes.

As indicated above, the polyolefin or polydiene chains can carry groups which can establish hydrogen interactions, in particular if the polyolefin grafts are not crystallizable. These groups which can establish hydrogen bonds are introduced into the grafts by copolymerization with appropriate monomers, in particular with monomers carrying —CO₂H groups, such as (meth)acrylic acid, crotonic acid, maleic anhydride or acid, itaconic acid or anhydride.

Mention may be made, as examples of semi-crystalline polyesters, of semi-crystalline polyesters such as those resulting from the polyesterification between diols and diacids, such as ethylene glycol, propylene glycol, diethylene glycol, neopentyl glycol, 1,4-butanediol, furandimethanol, cyclohexanedimethanol, glycerol, trimethylolpropane, pentaerythitol, and their mixtures, with polycarboxylic acids, in particular dicarboxylic acids, and their C₁-C₄ ester derivatives, for example succinic acid, glutaric acid and adipic acid or their dimethyl esters, phthalic anhydride or dimethyl terephthalate, or with lactones, for example caprolactone, or with cyclic dimers, such as polylactides or polyglycolides;
polyesteramides obtained by inclusion of amino alcohols, such as ethanolamine, in polyesterification mixtures.

Mention may be made, as examples of polyamides, of the polyamides obtained by condensation between an aliphatic, cycloaliphatic or aromatic C₃-C₅₀ dicarboxylic acid (or C₁-C₄ ester derivative) and a linear or branched aliphatic, cycloaliphatic or aromatic C₂-C₅₀ diamine, it being possible for the diacids to be chosen from the diacids mentioned above, with, in addition, dimeric fatty acids (originating from the condensation between two molecules of unsaturated fatty monoacids), it being possible for the diamines to be chosen from ethylenediamine, 1,2-diaminopropane, 1,3-diaminopropane, 1, 4-diamino-butane, 1,2-diamino-2-methylpropane, 1,6-diaminohexane, 1,10-diaminodecane, isophoronediamine, adamantane-diamine or 2,6-diaminopyridine.

Mention may be made, as examples of polyurethanes and polyureas, of the polyurethanes, polyureas and polyureas-urethanes obtained by polyaddition between aliphatic, cycloaliphatic and/or aromatic C₄-C₁₀₀, preferably C₄-C₃₀, diisocyanates, such as hexamethylene diisocyanate, isophorone diisocyanate, toluene diisocyanate or diphenylmethane diisocyanate, and diols, such as defined above, or diamines, such as defined above, or diols/diamine mixtures.

As regards the polycondensates comprising an X₁ reactive end, their preparation, in particular as regards polyesters and polyamides, does not require specific adjustment for the introduction of the reactive group in so far as the latter already exists at the chain end.

For example, a polyester generally comprises, by the end of the preparation, a reactive —CO₂H end and an —OH end. It should be noted that this —OH end will preferably be blocked by an inert group devoid of labile hydrogen, in order not to interfere with the grafting reaction with the starting cellulose derivative (nitrocellulose or cellulose ester).

The same comments are applicable for a polyamide, which exhibits a reactive —CO₂H end and an NH₂ end to be protected by a group which is inert with respect to the grafting reaction with a starting cellulose derivative.

It is also possible to introduce a reactive group X₁ into the polycondensate by introduction, into the reaction medium during polycondensation, of a reactant carrying the X₁ group, which must be inert with respect to the type of polycondensation chosen or inert under the experimental conditions of the polycondensation, and a single group capable of participating in the polycondensation. This reactant is thus monofunctional with respect to the polycondensation and thus acts as chain-limiting agent.

This reactant which is monofunctional with respect to the polycondensation and which carries a group which is reactive with respect to the hydroxyl functional groups of the starting cellulose derivative is preferably introduced during the polycondensation, so that the chains of the polymer are terminated only by a single reactive group X₁.

As regards the grafting of polyorganosiloxane chains, use may be made of a polymer comprising a reactive end, the backbone of which is polyorganosiloxane. Such polymers can be commercial polymers, such as the polydimethylsiloxanes sold by Shin-Etsu, the said polymers comprising a sequence of units: 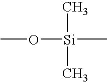
and a single reactive end of the —OH, epoxide or ethylene type.

The invention will now be described with reference to the following examples, given by way of illustration and without implied limitation.

### DETAILED DESCRIPTION OF SPECIFIC EMBODIMENTS

The following examples illustrate the preparation of cellulose derivatives according to the present invention and a formulation example comprising such cellulose derivatives.

### EXAMPLE 1

This example sets out the preparation of a cellulose acetatobutyrate comprising octadecyl carbamate side groups.

The reactants used are as follows:
100 g of cellulose acetatobutyrate CAB 553-0.4 from Eastman comprising 4.8% by weight of free hydroxyl groups;
10.4 g of octadecyl monoisocyanate;
1000 g of ethyl acetate;
1 g of tin 2-ethylhexanoate.

The procedure is as follows:
900 g of ethyl acetate and then, portionwise, 100 g of cellulose acetatobutyrate CAB 553-0.4 from Eastman are poured into a reactor equipped with a central stirrer, a thermometer, a reflux condenser, a system for sparging with nitrogen and a dropping funnel. The addition is subsequently carried out, with stirring and at reflux of the ethyl acetate, and then the reaction medium is brought back to a temperature of 25° C. once all the cellulose acetate/butyrate has dissolved. The octadecyl isocyanate, dissolved beforehand in 100 g of ethyl acetate, is then introduced into the funnel and the catalyst, tin 2-ethylhexanaoate, is added to the polymer solution. Subsequently, the isocyanate solution is added dropwise to the reactor at ambient temperature while maintaining stirring with nitrogen sparging. The mixture is subsequently heated at 55° C. with stirring and nitrogen sparging. These conditions are maintained for 8 hours.

The solution obtained is subsequently brought back to ambient temperature. Purification is carried out with precipitation of the solution from 10 ml of heptane. The precipitate formed is dried under vacuum. 105 g of cellulose acetate/butyrate comprising octadecyl carbamate side groups are thus obtained.

### EXAMPLE 2

A coloured nail varnish is prepared which has the following composition:

| | |
|---|---|
| Cellulose derivative of Example 1: | 3 g |
| Plasticizer: | 7 g |
| Isopropyl alcohol: | 5 g |
| Film-forming polymers: | 28 g |
| Pigments: | 1 g |
| Ethyl acetate/butyl acetate: | q.s. for 100 g |

The film-forming polymers are composed of a blend of nitrocellulose and of coresins.

After application of the nail varnish composition and after drying, a smooth, homogeneous and glossy film is obtained.

1. Use, as thickening agent for the organic phase of a nail varnish composition, of cellulose derivatives chosen from nitrocelluloses, cellulose esters, the said derivatives comprising free hydroxyl functional groups replaced, in all or part, by radicals of formula —OYR, in which:
R represents a group chosen from:
a) saturated or unsaturated hydrocarbonaceous groups comprising linear or branched chains or saturated or unsaturated cyclic hydrocarbonaceous groups,
it being possible for the said groups to comprise, in their chains, one or more aromatic groups and/or one or more heteroatoms chosen from O, N, P, Si or S;
b) fluoro- or perfluoroalkyl groups;
c) groups of polymeric nature chosen from polyolefins, polydienes or polycondensates;
d) organosiloxane or polyorganosiloxane groups;
e) mesogenic groups;
it being possible for the said groups corresponding to the definition a), b), c), d) or
e) to comprise at least one group capable of establishing a hydrogen bond,
Y represents a single bond or a divalent bonding group.

2. Use according to claim 1, in which the organic phase of the nail varnish composition comprises at least one ester solvent.

3. Use according to claim 1, in which the divalent bonding group Y is chosen from the —(C═O)—, —(C═O)O—, —SO₂—, —CO—NH—, —CO—NR′— or —Si(R₃)₂— groups, the R₃ groups, which are identical or different, being a linear or branched hydrocarbonaceous group comprising from 1 to 500 carbon atoms or a cyclic hydrocarbonaceous group comprising from 3 to 500 carbon atoms, the said group being saturated or unsaturated and being able to comprise one or more atoms from O, N, S, Si and/or P, and R′ denoting an alkyl radical comprising from 1 to 4 carbon atoms.

4. Use according to claim 1, in which the R group, when it corresponds to the definition a), is a linear or branched alkyl groups comprising from 1 to 50 carbon atoms, preferably from 8 to 50 carbon atoms.

5. Use according to claim 1, in which the R group, when it corresponds to the definition b), is a fluoro- or perfluoroalkyl group comprising from 6 to 50 carbon atoms.

6. Use according to claim 1, in which the R group, when it represents a polyolefin according to the definition c), is a polyolefin chosen from polyethylenes, (ethylene/propylene) copolymers, (ethylene/butene) copolymers, (ethylene/hexene) copolymers or (ethylene/octene) copolymers.

7. Use according to claim 1, in which the R group, when it represents a polycondensate according to the definition c), is chosen from polyesters, polyamides, polyesteramides, polyurethanes, polycarbonates, polyureas, (urea/urethane) copolymers or polyethers.

8. Use according to claim 1, in which, when R is a group corresponding to the definition d), it corresponds to the following formula:
—[Si(Ra,Rb)—O—]ₚ—Si(Ra,Rb,Rc),
in which:
the Ra, Rb and Rc groups, which are identical or different, represent a linear or branched alkyl chain comprising from 1 to 20 carbon atoms, a phenyl or a perfluoroalkyl chain comprising from 3 to 20 carbon atoms, and p is an integer ranging from 0 to 500.

9. Use according to claim 1, in which, when R is a group corresponding to the definition e), it is chosen from alkylaryl, alkylcycloalkyl, alkoxyaryl or alkoxycycloalkyl groups.

10. Use according to claim 9, in which R is chosen from the groups of the following formulae: 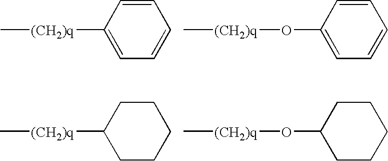
with q being an integer ranging from 1 to 10.

11. Use according to claim 1, in which, when R is a group corresponding to the definition e), it is represented by the following formula:
-E-F-E-G,
in which:
the E groups, which are identical or different, are divalent aromatic groups;
F is a divalent unsaturated and non-cyclic group or a single bond;
G represents an end group of following formulae:
—O—Ru, —CO—Ru, —CN, —COOH, —(CH₂)_{q}—Ru
with Ru representing a linear or branched alkyl group comprising from 1 to 10 carbon atoms and q corresponding to the definition given in claim 10.

12. Use according to claim 11, in which R is chosen from the groups of following formulae: 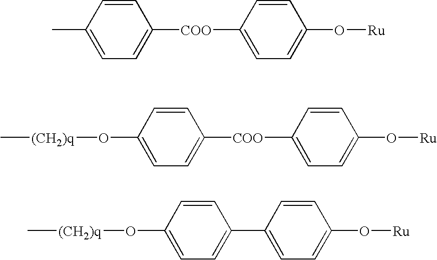
with Ru corresponding to the definition of claim 11 and q corresponding to the definition of claim 10.

13. Use according to claim 1, in which, when R is a group corresponding to the definition e), it represents a group derived from para-hydroxybenzoic acid corresponding to one of the following formulae: 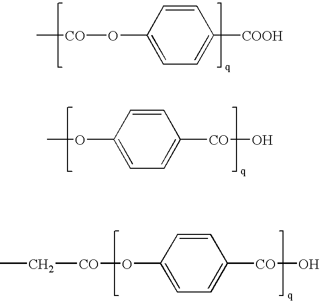
with q corresponding to the definition given in claim 10.

14. Use according to claim 1, in which the group capable of establishing a hydrogen bond is chosen from the groups of following formulae:
hydroxyl —OH;
carboxylic acid —COOH;
amino —NR₁R₂ with R₁ and R₂ identical or different;
pyridino of formula: 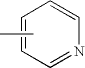
pyrimidino of formula: 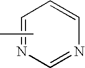
oxazolino corresponding to one of the following formulae: 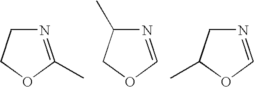
amido of formulae —NH—CO—R′ or —CO—NH—R₁;
pyrrolidono corresponding to one of the following formulae: 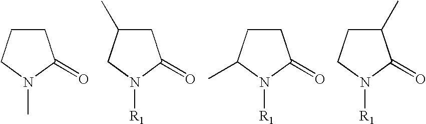
carbamoyl of formulae —O—CO—NH—R′ or —NH—CO—O—R′;
thiocarbamoyl of formulae —O—CS—NHR₁ or —NH—CS—O—R′;
carbonate —O—CO—O—R′;
ureyl —NR₁—CO—N(R₁)₂, the R₁ groups being identical or different;
thioureyl —NR₁—CS—N(R₁)₂, the R₁ groups being identical or different;
oxamido —NR₁—CO—CO—N(R₁)₂ with the R₁ groups identical or different;
guanidino —NH—C(═NH)—N (R₁)₂ with the R₁ groups identical or different;
biguanidino —NH—C(═NH)—NH—C(═NH)—N(R₁)₂ with the R₁ groups identical or different;
sulphonamido —NR₁—S(═O)₂—R′,
with R₁ and R₂ representing H or an alkyl group comprising from 1 to 4 carbon atoms, R′ representing an alkyl radical comprising from 1 to 4 carbon atoms.

15. Nail varnish composition comprising an organic phase based on at least one organic solvent and comprising at least one cellulose derivative comprising hydroxyl functional groups replaced, in all or part, by radicals of formula —OYR, in which:
R represents a group chosen from:
a) saturated or unsaturated hydrocarbonaceous groups comprising linear or branched chains or saturated or unsaturated cyclic hydrocarbonaceous groups,
it being possible for the said groups to comprise, in their chains, one or more aromatic groups and/or one or more heteroatoms chosen from O, N, P, Si or S;
b) fluoro- or perfluoroalkyl groups;
c) groups of polymeric nature chosen from polyolefins, polydienes or polycondensates;
d) organosiloxane or polyorganosiloxane groups;
e) mesogenic groups;
it being possible for the said groups corresponding to the definition a), b), c), d) or
e) to comprise at least one group capable of establishing a hydrogen bond,
Y represents a single bond or a divalent bonding group;
the said cellulose derivative being a cellulose ester when R corresponds to the definition a), c) and d), a cellulose ester or a nitrocellulose when R corresponds to the definition b), e).

16. Composition according to claim 15, in which the cellulose derivative or derivatives represent(s) from 0.1 to 60%, preferably from 0.5 to 40% and better still from 1 to 30% by weight, with respect to the total weight of the nail varnish composition.

17. Composition according to claim 15, in which the organic phase of the nail varnish composition comprises at least one ester solvent.

18. Composition according to claim 17, for which the ester solvent is an ester comprising from 3 to 8 carbon atoms.

19. Composition according to claim 15, in which the organic solvent or solvents are present at a content of 20 to 90%, preferably of 30 to 80% and better still of 40 to 70% by weight, with respect to the total weight of the composition.

20. Composition according to claim 15, in which the divalent bonding group Y is chosen from the —(C═O)—, —(C═O)O—, —SO₂—, —CO—NH—, —CO—NR′— or —Si(R₃)₂— groups, the R₃ groups, which are identical or different, being a linear or branched hydrocarbonaceous group comprising from 1 to 500 carbon atoms or a cyclic hydrocarbonaceous group comprising from 3 to 500 carbon atoms, the said group being saturated or unsaturated and being able to comprise one or more atoms from O, N, S, Si and/or P, and R′ denoting an alkyl radical comprising from 1 to 4 carbon atoms.

21. Composition according to claim 15, in which the R group, when it corresponds to the definition a), is a linear or branched alkyl groups comprising from 1 to 50 carbon atoms, preferably from 8 to 50 carbon atoms.

22. Composition according to claim 15, in which the R group, when it corresponds to the definition b), is a fluoro- or perfluoroalkyl group comprising from 6 to 50 carbon atoms.

23. Composition according to claim 15, in which the R group, when it represents a polyolefin according to the definition of c), is a polyolefin chosen from polyethylenes, (ethylene/propylene) copolymers, (ethylene/butene) copolymers, (ethylene/hexene) copolymers or (ethylene/octene) copolymers.

24. Composition according to claim 15, in which the R group, when it represents a polycondensate according to the definition c), is chosen from polyesters, polyamides, polyesteramides, polyurethanes, polycarbonates, polyureas, (urea/urethane) copolymers or polyethers.

25. Composition according to claim 15, in which, when R is a group corresponding to the definition d), it corresponds to the following formula:
—[Si(Ra,Rb)—O—]ₚ—Si(Ra,Rb,Rc),
in which:
the Ra, Rb and Rc groups, which are identical or different, represent a linear or branched alkyl chain comprising from 1 to 20 carbon atoms, a phenyl or a perfluoroalkyl chain comprising from 3 to 20 carbon atoms, and p is an integer ranging from 0 to 500.

26. Composition according to claim 15, in which, when R is a group corresponding to the definition e), it is chosen from alkylaryl, alkylcycloalkyl, alkoxyaryl or alkoxycycloalkyl groups.

27. Composition according to claim 26, in which R is chosen from the groups of following formulae: 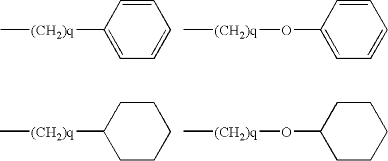
with q being an integer ranging from 1 to 10.

28. Composition according to claim 15, in which, when R is a group corresponding to the definition e), it is represented by the following formula:
-E-F-E-G,
in which:
the E groups, which are identical or different, are divalent aromatic groups;
F is a divalent unsaturated and non-cyclic group or a single bond;
G represents an end group of following formulae:
—O—Ru, —CO—Ru, —CN, —COOH, —(CH₂)_{q}—Ru
with Ru representing a linear or branched alkyl group comprising from 1 to 10 carbon atoms and q corresponding to the definition given in claim 27.

29. Composition according to claim 28, in which R is chosen from the groups of following formulae: 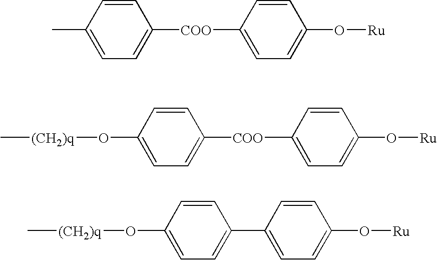
with Ru corresponding to the definition given in claim 28 and q corresponding to the definition given in claim 27.

30. Composition according to claim 15, in which, when R is a group corresponding to the definition e), it represents a group derived from para-hydroxybenzoic acid corresponding to one of the following formulae: 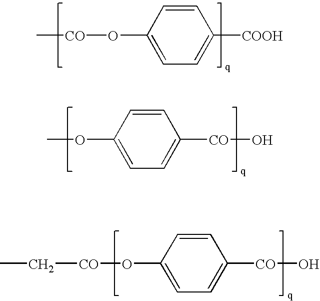
with q corresponding to the definition given in claim 27.

31. Composition according to claim 15, in which the group capable of establishing a hydrogen bond is chosen from the groups of following formulae:
hydroxyl —OH;
carboxylic acid —COOH;
amino —NR₁R₂ with R₁ and R₂ identical or different;
pyridino of formula: 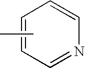
pyrimidino of formula: 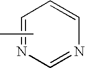
oxazolino corresponding to one of the following formulae: 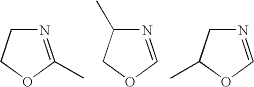
amido of formulae —NH—CO—R′ or —CO—NH—R₁;
pyrrolidono corresponding to one of the following formulae: 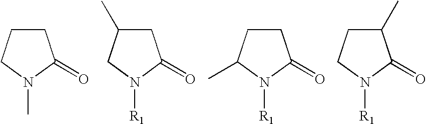
carbamoyl of formulae —O—CO—NH—R′ or —NH—CO—O—R′;
thiocarbamoyl of formulae —O—CS—NHR₁ or —NH—CS—O—R′;
carbonato —O—CO—O—R′;
ureyl —NR₁—CO—N(R₁)₂, the R₁ groups being identical or different;
thioureyl —NR₁—CS—N(R₁)₂, the R₁ groups being identical or different;
oxamido —NR₁—CO—CO—N(R₁)₂ with the R₁ groups identical or different;
guanidino —NH—C(═NH)—N(R₁)₂ with the R₁ groups identical or different;
biguanidino —NH—C(═NH)—NH—C(═NH)—N(R₁)₂ with the R₁ groups identical or different;
sulphonamido —NR₁—S(═O)₂—R′,
with R₁ and R₂ representing H or an alkyl group comprising from 1 to 4 carbon atoms, R′ representing an alkyl radical comprising from 1 to 4 carbon atoms.

32. Composition according to claim 15, additionally comprising one or more additives chosen from film-forming agents, plasticizers, colouring materials, such as pigments, pearlescent agents, glitter, thickening agents other than the cellulose derivatives as defined in claims 15 to 31, spreading agents, wetting agents, dispersing agents, antifoaming agents, preservatives, Uv-screening agents, active principles, surfactants, waxes, moisturising agents, fragrances, neutralizing agents, stabilizing agents or antioxidants.
